(19) United States
(12) Patent Application Publication
(10) Pub. No.: US 2007/0020312 A1
(43) Pub. Date: Jan. 25, 2007

(54) Method of fabricating a bioactive agent-releasing implantable medical device

(76) Inventors: Jessica Renee DesNoyer, San Jose, CA (US); Stephen Dirk Pacetti, San Jose, CA (US); Lothar Walter Kleiner, Los Altos, CA (US); Syed Faiyaz Ahmed Hossainy, Fremont, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/186,030

(22) Filed: Jul. 20, 2005

(51) Int. Cl. A61L 33/00

(52) U.S. Cl. 424/426; 427/2.24

(57) ABSTRACT

The present invention relates to methods of controlling the loading of a bioactive agent into a polymeric carrier to be coated on an implantable medical device to achieve controlled release of the bioactive agent.

### FIELD

This invention relates to the field of implantable medical devices (IMDs), more particularly to implantable medical devices having a coating from which bioactive agent(s) can be released at a target site in patient's body.

### BACKGROUND

The discussion that follows is intended solely as background information to assist in the understanding of the invention herein; nothing in this section is intended to be, nor is it to be construed as, prior art to this invention.

In the early 1980's, the utility of IMDs, which had been in use by the medical community for about 30 years, was expanded to include localized delivery of bioactive agents, specifically at the time, drugs. It was found that implantable devices could be fabricated with drugs incorporated directly into their structure or, more commonly, incorporated as a coating adhered to a surface of the IMD. In either case, the drug is shielded from the environment until the device is delivered to and released at the treatment site. The advantages of localized drug delivery are manifest.

Localized delivery permits the establishment of a high local concentration of a drug with concomitant low levels of systemic exposure and toxicity. In this manner, for example, the hemorrhagic complications that can accompany systemic delivery of an antithrombotic agent can be avoided. Likewise, the pervasive toxicity of antineoplastics to all living cells can be focused on malignant cells by delivery of the drug only at or into a tumor. Localized delivery also permits use of drugs that, for one reason or another, are not particularly amenable to delivery by other means. This includes drugs that, for instance, are susceptible to degradation under physiological conditions of temperature, pH, enzymatic activity, etc. and therefore would biodegrade before reaching the treatment site if administered systemically, and drugs that are so insoluble in physiological solution, which is primarily aqueous, that they precipitate and are immobilized almost immediately on administration. Of course, the ability to use less of a drug using localized delivery can also constitute a substantial economic advantage. Drugs can be transported to and released at desired treatment sites by a number of techniques.

For example, a drug can be coated per se on an implantable device and then over-coated with a layer of material that protects the drug layer but that either biodegrades in situ to release the drug, or is sufficiently permeable to bodily fluids to permit elution of the drug. A drug can be covalently bonded to a biodegradable polymer such that either the bond between the drug and the polymer is susceptible to biodegradation or, when the polymer degrades, the fragment left bonded to the drug has no affect on its pharmaceutical activity. One of the more common techniques for localized delivery is to simply disperse the drug in a polymeric carrier to create a “drug reservoir” from which the drug can be eluted once located at a treatment site. Each of the preceding techniques suffers from a variety of shortcomings; however, one that is particularly pervasive is control of the rate of release of the drug.

The rate of release of a drug from a IMD will influence both the local concentration of the drug and how long that concentration is maintained. This can be important because many drugs have a minimum effective concentration (MEC) below which they cannot exert their full therapeutic effect. Furthermore, the MEC often must be maintained for an extended period to achieve maximum effect. If the drug is released too rapidly from a device, it may reach or exceed its MEC quickly but be gone before it has had time to fully accomplish its task. By the same token, if release is too slow, the drug may be present for a long time but never at or above its MEC. Several factors affect the release rate of a drug from a reservoir. Prominent among these are drug loading and the composition of the reservoir. With regard to drug loading, not only is the amount of drug important, how the drug is loaded is also important.

Normally, to load a drug, the drug and a polymeric carrier are dissolved in a solvent or mixture of solvents, applied to an implantable device and the solvent is removed. When applied, the drug(s)/polymer(s) is(are) initially dispersed relatively evenly throughout the layer and thus the drug would be expected to be released at a fairly consistent rate over time from all regions of the layer. However, it is often the case that this initial homogeneity is upset during the drying process. That is, as the solvent moves to and evaporates from the surface of the drug-containing layer the drug migrates with it in chromatographic fashion and thus becomes concentrated near the surface of the layer. When the device is implanted and environmental conditions either erode the polymer or penetrate into it and elute the drug, the drug is released essentially en masse, an effect referred to as “burst” release. While burst-release may be desirable in some cases, for most drugs under most circumstances it is undesirable.

What is needed is a method of preparing a bioactive agent-releasing IMD wherein bioactive agent(s) is(are) essentially homogenously dispersed in a drug reservoir layer so that it(they) can be released at a substantially consistent rate in vivo. The present invention provides such a method.

### SUMMARY

Thus in one aspect, the present invention relates to a method of fabricating a bioactive agent-releasing implantable medical device, comprising:
providing an implantable medical device;
providing one or more polymer(s) each of which is less than about 50 wt % crystalline at 40° C.;
providing one or more bioactive agents;
providing a first solvent or mixture of two or more solvents, each of which
individually has a boiling point of about 100° C. or less at atmospheric pressure;
providing a second solvent that has, or mixture of two or more solvents each of which individually has, a boiling point at atmospheric pressure greater than 100° C. and at least one of which has a boiling point at atmospheric pressure that is at least 25° C. higher than the highest boiling first solvent at atmospheric pressure;
   wherein:
each bioactive agent is at least 10% wt % soluble in the first solvent or each solvent of the first mixture of solvents; and,
each bioactive agent is less that 10% wt % soluble in the second solvent or each solvent of the second mixture of solvents;
dissolving the polymer(s) and bioactive agent(s) in a mixture of the first and the second solvent(s) at a ratio of first solvent(s) to second solvent(s) that results in a homogenous solution;
applying a layer of the homogenous solution to the medical device; and,
drying the layer of homogeneous solution to form a bioactive agent reservoir layer.

In an aspect of this invention, each polymer is less than or equal to 30 wt % crystalline at 40° C.

In an aspect of this invention, each polymer is less than or equal to 20 wt % crystalline at 40° C.

In an aspect of this invention, each bioactive agent is less than 5 wt % soluble in the second solvent or each solvent of the second mixture of solvents.

In an aspect of this invention, each bioactive agent is less than 1 wt % soluble in the second solvent or each solvent of the second mixture of solvents.

In an aspect of this invention, at least one of the polymers is a poly(ester-amide).

In an aspect of this invention, the poly(ester-amide) comprises:
one or more amino acid-based constitutional units;
one or more diol-based constitutional units; and,
one or more diacid-based constitutional units.

In an aspect of this invention, if an amino acid-based constitutional unit is enantiomeric, the ratio of D-amino acid to L-amino acid for each enantiometic constitutional unit is independently from about 30:70 to about 70:30.

In an aspect of this invention, the ratio of D-amino acid to L-amino acid for each enantiomeric constitutional unit is about 50:50, that is, the constitutional unit is a racemate.

In an aspect of this invention, the amino-acid-based consititutional unit(s) is(are) derived from L-amino acid(s).

In an aspect of this invention, the amino acid-based constitutional units is (are) derived from monomers selected from the group consisting of glycine, valine, alanine, leucine, isoleucine, lysine, tyrosine, glutamic acid, cysteine and phenyalanine.

In an aspect of this invention, the diol monomer-based constitutional unit(s) is (are) derived from monomers selected from the group consisting of (2C-12C)alkyldiol, (3C-8C)cycloalkyldiol; (4C-12C)alkenyldiol and (4C-12C)alkynyldiol.

In an aspect of this invention, the diol-based constitutional unit(s) is (are) derived from monomers selected from the group consisting of poly(ethylene glycol), poly(propylene glycol) and hydroxy-terminated PVP.

In an aspect of this invention, the diacid-based constitutional units is (are) derived from monomers selected from the group consisting of (0C-12C)alkyldiacid, (2C-12C)alkyenyldiacid, (2C-12C)alkynyldiacid and aryldiacid.

In an aspect of this invention, the monomers is (are) selected from the group consisting of oxalic acid, maleic acid, malonic acid, succinic acid, adipic acid, sebacic acid, terephthalic acid and isophthalic acid.

In an aspect of this invention, the polymer is selected from the group consisting of poly(L-lactide), poly(D-lactide), poly(D,L-lactide), poly(meso-lactide), poly(L-lactide-co-glycolide), poly(D-lactide-co-glycolide), poly(D,L-lactide-co-glycolide) and poly(meso-lactide-co-glycolide), wherein the ratio of D-lactide to L-lactide in the D,L-lactide is from about 5:95 to about 95:5.

In an aspect of this invention, the ratio of D-lactide to L-lactide in the D,L-lactide is about 50:50, that is, the D,L-lactide is racemic.

In an aspect of this invention, one or more of the first solvent(s), the second solvent(s) or both is(are) hydroscopic; and the homogenous solution is applied to the implantable medical device in an at least 40% relative humidity environment, wherein: each bioactive agent is less than 10 wt % soluble in water and each polymer is at least 10% wt % soluble in water.

In an aspect of this invention, the first and second solvent or mixture of solvents are identical, that is, there is effectively only one solvent or mixture of solvents and one or more of the solvent(s) is(are) hygroscopic.

In an aspect of this invention, each bioactive agent is less than 5% wt % soluble in water.

In an aspect of this invention, each bioactive agent is less than w/w 1 wt % soluble in water.

In an aspect of this invention, the method herein further comprises:
providing one or more topcoat polymer(s);
dissolving the topcoat polymer(s) in a solvent or mixture of solvents to form a homogenous solution;
applying the homogenous solution to the bioactive agent reservoir layer to form a solvent-containing topcoat polymer layer; and,
drying the solvent-containing polymer layer to form a topcoat layer.

In an aspect of this invention, each bioactive agent is at least 10 wt % soluble in the solvent or mixture of solvents used to dissolve the topcoat polymer(s).

In an aspect of this invention, each bioactive agent is less than 10 wt % soluble in the solvent or in the mixture of solvents used to dissolve the topcoat polymer(s).

In an aspect of this invention, each bioactive agent is less than 5 wt % soluble in the solvent or mixture of solvents used to dissolve the topcoat polymer(s).

In an aspect of this invention, each bioactive agent is less than 1 wt % soluble in the solvent or mixture of solvents used to dissolve the topcoat polymers.

In an aspect of this invention, the topcoat polymer(s) is (are) selected from the group consisting of poly(L-lactide), poly(D-lactide), poly(D,L-lactide), poly(meso-lactide), poly(D,L-lactide-block-ethylene glycol-block-D,L-lactide), and poly(meso-lactide-block-ethylene glycol-block-meso-lactide) wherein:

the ratio of D-lactide to L-lactide in the D,L-lactic acid for each polymer is independently from about 30:70 to about 70:30.

In an aspect of this invention, the method herein further comprises poly(ethylene glycol) blended with the indicated polymer(s) wherein the poly(ethylene glycol) has an average molecular weight of about 1,000 Da to about 30,000 Da.

In an aspect of this invention, the method herein further comprises poly(ethylene glycol-bl-propylene glycol-bl-ethylene glycol) (Pluronic™) wherein the Pluronic™ has an average molecular weight of less than 30,000 Da.

In an aspect of this invention, the ratio of D-lactide to L-lactic acid in each D,L-lactic acid-containing polymer is about 50:50.

In an aspect of this invention, the topcoat polymer is poly(D,L-lactic acid).

In an aspect of this invention, the poly(D,L-lactide) topcoat polymer comprises acid end groups.

In an aspect of this invention, the topcoat polymer when dried forms a topcoat layer having a thickness of from about 0.1 to 20 microns.

In an aspect of this invention, the poly(D,L-lactide) has an average molecular weight of from about 20,000 Da to about 500,000 Da.

In an aspect of this invention, the poly(D,L-lactide has an average molecular weight of from about 20,000 Da to about 100,000 Da.

In an aspect of this invention, the method herein further comprises a plasticizer.

In an aspect of this invention, the plasticizer comprises poly(D,L-lactide) having an average molecular weight of about 2,000 Da to about 20,000 Da.

In an aspect of this invention, the method herein further comprises a porogen.

In an aspect of this invention, the bioactive agent comprises one or more of a therapeutic agent, a prophylactic agent and/or a diagnostic agent.

In an aspect of this invention, the therapeutic or prophylactic agent is selected from the group consisting of an antiproliferative, an antineoplastic, an antiplatelet, an anticoagulant, an antifibrin, an antithrombotic, a cytostatic and an antiallergenic.

In an aspect of this invention, the therapeutic or prophylactic agent is selected from the group consisting of tacrolimus, clobestasol, dexamethasone, rapamycin, 40-O-(2-hydroxyethyl)rapamycin, 40-O-(3-hydroxypropyl)rapamycin, 40-O-[2-(2-hydroxyethoxy)]ethylrapamycin and 40-O-tetrazolylrapamycin.

### DETAILED DESCRIPTION

In the discussion that follows, it is understood that, with regard to various aspects of this invention, singular implies plural and visa versa. For example, “a bioactive agent” or “the bioactive agent” refers to a single bioactive agent or to a plurality of bioactive agents; “a polymer” or “the polymer” refers to a single polymer or a plurality of polymers, etc.

As used herein, an IMD refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of IMDs include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves and cerebrospinal fluid shunts. Of course, an IMD specifically designed and intended solely for the localized delivery of a bioactive agent is within the scope of this invention. The IMD may be constructed of any biocompatible material capable of being coated with an adherent layer containing a bioactive agent.

For example, an IMD useful with this invention may be made of one or more biocompatible metals or alloys including, but not limited to, cobalt chromium alloy (ELGILOY, L-605), cobalt nickel alloy (MP-35N), 316 L stainless steel, high nitrogen stainless steel, e.g., BIODUR 108, nickel-titanium alloy (NITINOL), tantalum, platinum, platinum-iridium alloy, gold and combinations thereof.

Alternatively, the IMD may be made of one or more biocompatible, relatively non-biodegradable polymers including, but not limited to, polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins.

If desired, the IMD may be made of one or more naturally-occurring—and, therefore, inherently biocompatible and biodegradable—polymers including, without limitation, collagen, chitosan, alginate, fibrin, fibrinogen, cellulosics, starches, dextran, dextrin, hyaluronic acid, heparin, glycosaminoglycans, polysaccharides and elastin.

One or more synthetic or semi-synthetic biocompatible, biodegradable polymers may also be used to fabricate an IMD useful with this invention. As used herein, a synthetic polymer refers to one which is created entirely in the laboratory while a semi-synthetic polymer relates to a naturally-occurring polymer that has been modified in the laboratory. Examples of biodegradable synthetic polymers include, without limitation, polyphosphazines, polyphosphoesters, polyphosphoester urethane, polyhydroxyacids, polyhydroxyalkanoates, polyanhydrides, polyesters, polyorthoesters, poly(amino acids), polyoxymethylenes, poly(ester-amides) and polyimides.

Of course, blends of, and copolymers base on, any of the above may be used as well. Based on the disclosure herein, those skilled in the art will readily recognize those IMDs and those materials from which they can be fabricated that will be useful with this invention.

As used herein, “biocompatible” refers to a polymer that both in its intact, that is, as synthesized, state and in its decomposed state, i.e., its degradation products, is not, or at least is minimally, toxic to living tissue; does not, or at least minimally and reparably does, injure living tissue; and/or does not, or at least minimally and/or controllably does, cause an immunological reaction in living tissue.

As used herein, “biodegradable” refers to a polymer that has functional groups in its primary backbone that are susceptible to cleavage, usually but not necessarily, hydrolytic cleavage, when placed in a physiological milieu, that is, a primarily aqueous solution at pH approximately 7-7.5 usually in the presence of one or more hydrolytic enzymes or other endogenous biological compounds that catalyze or at least assist in the degradation process.

As used herein, a “bioactive agent-releasing” IMD refers to any appliance that contains within its structure or, more commonly, in a layer coated on all or a portion of its surface, a bioactive agent such that, when the device or layer is exposed to a physiological environment, the bioactive agent is released into the environment.

As used herein, a “homogeneous” solution or layer refers to a solution or layer in which a solute or dispersant is relatively uniformly dispersed throughout a solvent or dispersing medium such that a sample taken from anywhere in the solution or the layer will have the same composition as a sample taken from anywhere else in the solution or layer.

A presently preferred implantable medical device for use with the method of this invention is a stent. The stent may be self-expandable or balloon expandable. Any type of stent currently known, or as may become known, to those skilled in the art may be used with the method of this invention. A particularly useful purpose of a stent is to maintain the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, re-stenosis and vulnerable plaque Vulnerable plaque (VP) is a type of fatty build-up in an artery thought to be caused by atherosclerosis and inflammation. The VP is covered by a thin fibrous cap that can rupture leading to blood clot formation. A stent, the primary function of which is any of the above, may also be coated according to this invention so as to deliver bioactive agent(s) as well. Or the primary use of the stent so coated may in fact be localized delivery of a bioactive agent to a selected treatment site in a patient's body.

The polymers used in the reservoir layer of this invention are less than 50 weight percent (wt %), preferably less than less than 30 wt % and presently most preferably less than 20 wt %, crystalline at temperatures up to and including approximately 40° C. Crystallinity refers to regions of a bulk polymer where portions of the polymer chain or of portions of a number of separate chains align in a regular pattern. Regions of the bulk polymer that are not so aligned, that is, wherein the polymer chains are in an essentially random orientation with regard to one another, are said to be amorphous. Polymers having both amorphous and crystalline domains are said to be “semi-crystalline.” Determination of the weight percent of a polymer that is crystalline is relatively straight-forward and well-known to those skilled in the art.

Briefly, differential scanning calorimetry may used to determine the total heat of crystallization, T_{c}, and total heat of melting, Tₘ of a partially crystalline polymer. Tₘ-T_{c} provide the amount of heat given off by the sample before it was heated above T_{c}. Dividing (Tₘ-T_{c}) by the specific heat of melting Tₛₚ, i.e., the amount of heat required to melt one gram of the polymer, gives the total number of grams of the sample that were crystalline below T_{c}. Dividing this number by the total weight of the sample provides the weight percent of the polymer that is crystalline below the heat of crystallization.

While any biocompatible polymer that meets the above wt % crystallinity may be used in the method of this invention, at present it is preferred that at least one of polymers be a poly(ester-amide), a poly(lactide) or a poly(lactide-co-glycolide) copolymer.

Presently preferred poly(ester-amide)s are those comprised of: (1) an unsubstituted or substituted (0C-12C)diacid; (2) an unsubstituted or substituted naturally-occurring L-amino acid, the D-enantiomer of an unsubstituted or substituted naturally-occurring L-amino acid, a mixture of the foregoing L and D enantiomers, and/or an unsubstituted or substituted synthetic α-aminoacid; and (3) an unsubstituted or substituted (2C-12C)diol or polymeric diol such as poly(ethylene glycol) or poly(propylene glycol). The poly(ester-amide) may also optionally comprise an unsubstituted or substituted (2C-12C)diamine.

A generalized chemical formula of a poly(ester-amide) useful in the method of this invention is:

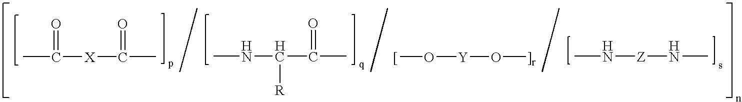

In the above formula, p, q, r and s refer to the mol fraction of each constitutional unit in the polymer. Thus p+q+r+s must equal unity, 1. Multiplying the mol fraction by 100 gives the mol percent (mol %) of each constitutional unit. The value of each individual variable can varied as desired, the only requirement being that sufficient molar quantities of each is present to form the requisite ester and amide bonds necessary to create the poly(ester-amide). In some structural representations of poly(ester amides), each of the above constitutional units may not be separately presented, i.e., one or more of them may be combined. Such will become apparent in the non-limiting examples that follow.

As used herein, a “constitutional unit” of a polymer refers to an iterating group of a polymer. For example, in the above poly(ester-amide) formula, —NHCH(R)C(═O)— is a constitutional unit that is derived from the amino acid monomer, RCH(NH₂)C(═O)OH.

The value of n is presently preferably between 20 KDa and 500 KDa (number average molecular weight). With regard to the molecular weights of polymers herein, if the polymer is a commodity, the molecular weight provided by the supplier is used without particular knowledge as to the method of its determination (unless, of course, the supplier indicates a method). With regard to polymers prepared ab initio herein, molecular weight refers to a number average molecular weight as determined by size exclusion chromatography.

As used herein, “weight percent” (wt %) refers to the portion of the weight of any material that can be attributed to a discrete sub-portion of that material, expressed as a percent. Thus, if a solute is described as being 10 wt % soluble in a solvent, it means that, at saturation, the percentage of the total weight of the saturated solution attributable to the solute is 10%. For example, if a salt is said to be 10% soluble in a solvent, then 10 grams of the salt would dissolve in 90 grams of the solvent. The total weight is then 100 grams of which 10 grams, or 10%, is salt. Weight percent crystallinity of a polymer is discussed above.

The poly(ester-amide) may be a random or block copolymer, as those terms are understood by those skilled in the art, so long as each bond between any two of the constitutional units is either an ester or an amide bond. X, Y and Z may be any chemical entity that results in a poly(ester-amide) that is biocompatible and within the parameters of this invention with regard to crystallinity. Presently preferred X, Y and Z groups are branched or unbranched (1C-20C)alkyl. Presently preferred diacids include one or more of (0C-12C)alkyl diacids, (2C-12C)alkenyl diacids, (2C-12C) alkynyl diacids and aryl diacids. Presently preferred amino acids include one or more of glycine, valine, alanine, leucine, isoleucine, phenylalanine, lysine, tyrosine, glutamic acid and cysteine. Presently preferred diols include one or more of (2C-12C)alkyl diol, (4C-12C)alkenyl diol, (4C-12C)aklynyl diol, poly(ethylene glycol), poly(propylene glycol) and hydroxyl-terminated poly(vinylpropylene).

As used herein, alkyl refers to a straight or branched chain, unsubstituted or substituted fully saturated (no double or triple bonds) hydrocarbon. The designation (m₁C-m₂C)alkyl means that the alkyl group contains from m₁ to and including m₂ carbon atoms in the chain. For example, a (2C-4C)alkyl refers to any one of CH₃, CH₃CH₂—, CH₃CH₂CH₂—, (CH₃)₂CH—, CH₃CH₂CH₂CH₂CH₂—, CH₃CH₂CH(CH₃)CH₂— or (CH₃)₃C—. As used herein, alkenyl refers to an alkyl that has one or more double bonds in the hydrocarbon chain while alkynyl refers to an alkyl that has one or more triple bonds in the hydrocarbon chain. A cycloalkyl group refers to an alkyl group in which the terminal carbon atoms of the hydrocarbon chain are joined to one another to form a ring. A diacid refers to a HOOC—X—COOH compound wherein X is —(CH₂)ₐ—SO that a “0C” alkyl means that a is 0 and the diacid is HOOC—COOH (oxalic acid) and a “2C” alkyl diacid would be HOOCCH₂CH₂COOH. As used herein, an aryl diacid refers to a phenyl or naphthyl diacid, in particular at present isophthalic and terephthalic acid.

As noted, the constitutional units herein may be unsubstituted or substituted. If substituted, the substituent is selected from the group consisting of any entity that will result in a biocompatible polymer or biodegradation fragment thereof. Presently preferred substituent groups are fluorine, chlorine and (1C-4C)alkyl groups.

An example, without limitation, of a poly(ester-amide) useful in the method of this invention is poly{[N,N′-sebacoyl-bis-(L-leucine)-1,6-hexylene diester]ₚ-co-[N,N′-sebacoyl-L-lysine benzyl ester]_{q}}ₙ:

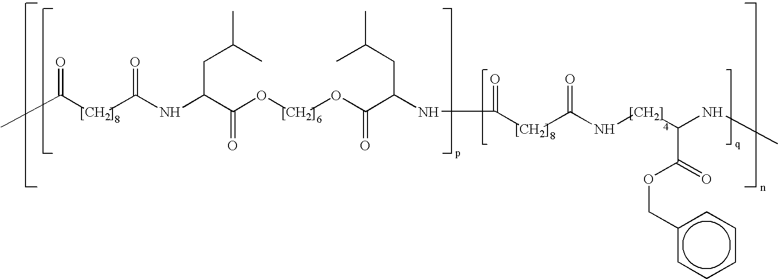

The mol fraction of p can range from 0.01 to 0.99, with q being (1.0-p).

Another example, without limitation, of a poly(ester-amide) useful in the methods of this invention is poly{[N,N′-sebacoyl-bis-(L-leucine)-1,6-hexylene diester]ₚ-co-[N,N′-sebacoyl-L-lysine 4-amino-TEMPO amide]_{q}}ₙ:

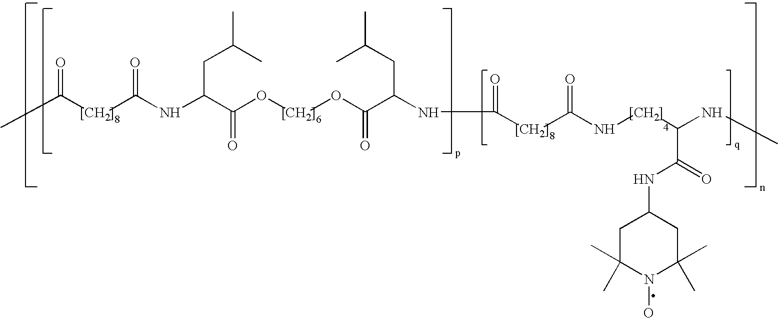

Again, the mol fraction of p can range from 0.01 to 0.99 and q=1.0-p.

Still other poly(ester-amide)s useful herein include those of simpler structure, consisting of one type of repeating block such as, without limitation, poly-[N,N′-sebacoyl-bis-(L-phenylalanine)-1,6-hexylene diester]ₙ:

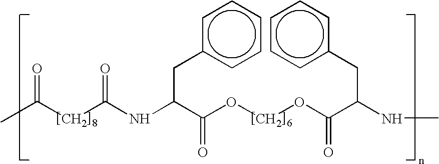

A further nom-limiting example of a “simpler” poly(ester-amide) is poly-[N,N′-succinyl-bis-(L-glycine)-1,3-propylene diester]ₙ:

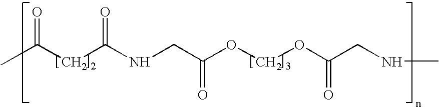

Presently preferred poly(lactide)s for use with the method of this invention include poly(L-lactide), poly(D-lactide), poly(D,L-lactide), poly(meso-lactide), and copolymers of any of the foregoing with glycolide. Meso-lactide refers to a cyclic lactide prepared from one molecule of L-lactic acid and one molecule of D-lactic acid. While the chemical composition of poly(D,L-lactide) and poly(meso-lactide) are identical, their morphology is different, with poly(meso-lactide) having no more than two consecutive L- or D-constitutional units while poly(D,L-lactide) has a statistical distribution of 2, 3, 4 and higher consecutive enantio-identical constitutional units.

As used herein, an enantiomer refers to an optically active compound, that is, an entity that contains at least one asymmetric carbon atom such that, when plane polarized light is shone through a solution of the compound, the light rotates either to the left (L, levorotary) or right (D, dextrorotary). A racemate refers to a 50:50 mixture of D and L enantiomers of a compound, which, in solution, results in 0 rotation (more accurately, L-rotation is exactly cancelled by D-rotation) of plane polarized light.

Any bioactive agent amenable to localized delivery may be used in the method of this invention. By “bioactive agent” is meant any substance that is of medical or veterinary therapeutic, prophylactic or diagnostic utility. By “amenable to” localized delivery is meant that the bioactive agent is sufficiently stable to withstand the formulation procedures employed to fabricate an IMD coated with a bioactive agent-releasing layer of this invention, is sufficiently stable to remain intact in the layer until delivered to the site of release and is capable of being released from the coating layer under physiological conditions of temperature, pH, ionic strength, etc.

As used herein, a therapeutic agent refers to a bioactive agent that, when administered to a patient, will cure, or at least relieve to some extent, one or more symptoms of, a disease or disorder.

As used herein, a prophylactic agent refers to a bioactive agent that, when administered to a patient either prevents the occurrence of a disease or disorder or, if administered subsequent to a therapeutic agent, prevents or retards the recurrence of the disease or disorder.

Bioactive agents that may be used in the method of this invention include, without limitation:
antiproliferative drugs such as actinomycin D, or derivatives or analogs thereof. Actinomycin D is also known as dactinomycin, actinomycin IV, actinomycin I₁, actinomycin X₁, and actinomycin C₁;
antineoplastics and/or antimitotics such as, without limitation, paclitaxel, docetaxel, methotrexate, azathioprine, vincristine, vinblastine, fluorouracil, doxorubicin hydrochloride, and mitomycin;
antiplatelet, anticoagulant; antifibrin, and antithrombin drugs such as, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin, and thrombin;
cytostatic or antiproliferative agents such as, without limitation, angiopeptin;
angiotensin converting enzyme inhibitors such as captopril, cilazapril or lisinopril;
calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide;
antiallergic agent such as, without limitation, permirolast potassium.
   other therapeutic agents such as, without limitation, alpha-interferon, genetically engineered epithelial cells, tacrolimus, clobetasol, dexamethasone and its derivatives, and rapamycin, its derivatives and analogs such as 40-O-(2-hydroxyethyl)rapamycin (EVEROLIMUS®), 40-O-(3-hydroxypropyl)rapamycin, 40-O-[2-(2-hydroxyethoxy)]ethyl-rapamycin, and 40-O-tetrazolylrapamycin.

If desired, the method of this invention may further comprise including a biobeneficial agent in the coating layer in addition to a bioactive agent. A biobeneficial agent is one that beneficially affects an IMD by, for example reducing the tendency of the device to protein foul, increasing the hemocompatibility of the device, and/or enhancing the non-thrombogenic, non-inflammatory, non-cytotoxic, non-hemolytic, etc. characteristics of the device, all without the intended release of any bioactive agent into the environment.

Representative biobeneficial materials include, but are not limited to, polyethers such as poly(ethylene glycol) (PEG) and poly(propylene glycol); copoly(ether-esters) such as poly(ethylene oxide-co-lactic acid); polyalkylene oxides such as poly(ethylene oxide) and poly(propylene oxide); polyphosphazenes, phosphorylcholine, choline, polymers and co-polymers of hydroxyl bearing monomers such as hydroxyethyl methacrylate, hydroxypropyl methacrylate, hydroxypropylmethacrylamide, poly(ethylene glycol)acrylate, 2-methacryloyloxyethylphosphorylcholine (MPC) and n-vinyl pyrrolidone (VP); carboxylic acid bearing monomers such as methacrylic acid, acrylic acid, alkoxymethacrylate, alkoxyacrylate, and 3-trimethylsilylpropyl methacrylate; polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functionalized poly(vinyl pyrrolidone); biomolecules such as fibrin, fibrinogen, cellulose, starch, collagen, dextran, dextrin, hyaluronic acid, heparin, glycosamino glycan, polysaccharides, elastin, chitosan, alginate, silicones, PolyActive™, and combinations thereof. PolyActive™ refers to a block copolymer of poly(ethylene glycol) and poly(butylene terephthalate).

The amount of bioactive agent in a coating will depend on the required MEC of the agent and the length of time over which it is desired that the MEC, or above, be maintained. For most bioactive agents the MEC will be known to, or readily derivable by, those skilled in the art from the literature. For experimental bioactive agents or those for which the MEC by localized delivery is not known, such can be empirically determined using techniques well-known to those skilled in the art.

As used herein, a “patient” refers to any organism that can benefit from the use of a bioactive agent releasing IMD. In particular at present, patient refers to a mammal such as a cat, dog, horse, cow, pig, sheep, rabbit, goat or, most preferably at present, a human being.

The method of this invention comprises the use of a solvent system in which the bioactive agent has differential solubility in the solvents used. In general, a relatively low boiling solvent in which the bioactive agent is relatively soluble and a relatively high boiling solvent in which the bioactive agent is relatively non-soluble are used. It is presently preferred that the polymer used be soluble in the high boiling solvent in order to facilitate overall formation of a reservoir layer.

The first solvent or mixture of solvents is one: (1) in which each individual solvent has a boiling point of 100° C. or below at atmospheric pressure and (2) in which each bioactive agent is at least 10 wt % soluble. The solvents should be miscible with one another and with the second solvent or mixture of solvents. Useful first solvents include, but are not limited to, hydrocarbons including, without limitation, pentane, hexanes, heptane, octane, cyclopentane, cyclohexane, petroleum ethers and benzene; chlorinated hydrocarbons including, without limitation, dichloromethane, dichloroethane, 1,1,1-trichloroethane, trichloroethylene, chloroform and carbon tetrachloride; and oxygenated solvents including, without limitation, ethers such as diethyl ether, diisopropyl ether, methyl t-butyl ether, tetrahydrofuran and dioxolane; ketones such as acetone and methyl ethyl ketone; alcohols such as methyl alcohol, ethyl alcohol, n-propyl alcohol, isopropyl alcohol, and tert-butyl alcohol; and esters such as methyl acetate and ethyl acetate.

The second solvent or mixture of solvents is one (1) in which each individual solvent has a boiling point over 100° C. at atmospheric pressure and (2) in each of which each bioactive agent is less than 10 wt % soluble. The solvents should be miscible with one another and with the first solvent or mixture of solvents. It is presently preferred that at least one of the second solvents have a boiling point that is at least 25° C. higher than that of the highest boiling first solvent. Examples of second solvents include, but are not limited to, dimethylformamide, dimethylacetamide, dimethyl sulfoxide, octane, nonane, cyclohexanol, cyclohexanone, 1,1,2-tricholorethane, dioxane, toluene, xylene, and white spirits (hydrocarbon fraction boiling between about 140° C. and 225° C.).

A general procedure for preparing the polymer/solvent/bioactive agent solution for coating an IMD would be to, first, determine the quantity of polymer or polymers that will be used in the coating. This will involve a calculation based on the area of the region(s) of the device to be coated, the desired coating thickness and desired coating density, that is, quantity of polymer per square inch. The amount of bioactive agent to be used must also be ascertained. This will require a determination of the MEC to be achieved at the release site and period of time that concentration is to be maintained. Once these have been determined, the desired amount of polymer is dissolved in the second solvent or mixture of solvents, the amount of solvent used being sufficient to just create a homogenous solution. The bioactive material is added to the solution. Due to the low solubility of the bioactive material in the second solvent and the fact that the second solvent is essentially saturated with polymer, a homogeneous solution should not be obtained. The first solvent or mixture of solvents, in which the bioactive material is more soluble, is then added until a homogeneous solution is obtained. The homogeneous solution is coated on an IMD using any technique known or as may become known to those skilled in the art. For example, without limitation, the solution may be applied by dipping, spraying, roll coating, brushing, and direct application by droplets. The coating is then dried at a temperature that is slightly below the boiling point of the first solvent or, in the case of a mixture of solvents, slightly below the boiling point of the lowest boiling of the first mixture of solvents. If a mixture of solvents is used, after the lowest boiling solvent has evaporated, the temperature may be increased to just under the boiling point of the next lowest boiling of the first mixture of solvents and so on until each solvent of the mixture of solvents has been removed. The procedure is then repeated to remove the second solvent or mixture of solvents. To keep the overall temperature to which the coating is exposed low enough to not adversely affect the incorporated bioactive agent (and any biobeneficial agent that might also be incorporated), the drying procedure may be include the use of a vacuum, care being taken to not apply a vacuum that causes any of the solvents to vaporize so rapidly at the reduced pressure so as to form bubbles in and disrupt the integrity of the coating.

In an aspect of this invention, the first solvent or mixture of solvents, the second solvent or mixture of solvents, or both may constitute one or more hygroscopic solvents. As used herein, a hygroscopic solvent refers to a solvent that, when exposed to a high humidity environment will absorb up to about 5 wt % water.

If hygroscopic solvents are used, the bioactive agent(s) should each be less than 10 wt % soluble in water and each polymer should be greater than 10 wt % soluble in water. The coating of the implantable medical device is them carried out in an atmosphere that has a relative humidity of about 40% or higher. As used herein, relative humidity refers to the amount of atmospheric moisture present relative to the amount that would be present if the air were saturated with water. As the coating is applied, the solvents absorb water from the air and, since the bioactive agent is selected to be minimally soluble in water, it precipitates from solution and is immobilized in the coated layer. Since precipitation and immobilization results before any substantial chromatographic movement can occur as the result of the drying process, the bioactive agent is relatively homogeneously dispersed in the layer and therefore will be released from the layer at a substantially constant rate.

In aspects of the invention involving use of a hydroscopic solvent, the first and second solvents may be identical. i.e., one solvent or mixture of solvents may be used and, as used herein, would constitute the “first solvent or mixture of solvents” while the absorbed water, in which the bioactive agent is insoluble, would constitute the “second solvent or mixture of solvents.” If this is the case, the solvent used should have a boiling point less than that of water, i.e., less than 100° C. at atmospheric pressure.

If desired, the IMD may further comprise a topcoat layer in addition to the reservoir layer. As used herein, a topcoat layer refers to a thin layer of initially non-bioactive agent-containing polymeric material that is coated atop the reservoir layer, that is, between the reservoir layer and the environment. As used herein, a thin layer refers to a layer that has a thickness of from about 0.1 to about 20 microns. The topcoat provides additional control of the rate of release of the bioactive material. There are several ways of topcoats may accomplish this added control.

For example, a topcoat of poly(lactide) may be used. The polylactide may be poly(L-lactide), poly(D-lactide), poly(D,L-lactide), poly(meso-lactide) or a combination thereof. In one aspect the polylactide layer is applied as a very thin layer, from about 0.1 microns to about 20 microns, that will biodegrade rapidly but which will provide a delayed onset of bioactive agent release. To avoid extraction of bioactive agent into the topcoat, the solvent used to prepare the topcoat coating solution should be such that the bioactive agent is less than about 10 wt % soluble in it. On the other hand, if an initial burst release of bioactive agent followed by timed release of the remainder of the agent is desired, the polylactide coating solution may comprise a solvent or solvents in which the bioactive agent is more than 10 wt % soluble. After coating, the solvent is removed slowly so as to give it time to extract a quantity of the drug into the topcoat layer from the reservoir layer at the interface of the two layers. The extracted bioactive agent will then be burst-released from the polylactide when it biodegrades while the remainder of the bioactive agent will be released from the reservoir over time.

The polylactide can be a blend of low molecular weight (about 2000-20,000 Da) with high molecular weight (greater than about 60,000 Da) polymers. The low molecular weight polymer will have a lower glass transition temperature (T_{g}) than the high molecular weight polymer, with the T_{g} of the blend being somewhere in-between. A polymer or blend above its T_{g} will be more permeable than a polymer below its T_{g} and will release a drug more readily by elution. By varying the amounts of the high and low T_{g} polymers, the blend can be tailored to a T_{g} that will provide the desired release kinetics. At present, an overall T_{g} of the blend that is below the body temperature of the patient is preferred. The low molecular weight polymer may be low molecular weight poly(lactide) (MW 2,000-30,000 Da), low molecular weight poly(ethylene glycol) (MW 1,000-30,000 Da), low molecular weight poly(vinylpyrrolidone) (MW less than 30,000 Da) or low molecular weight Pluronic™ (MW less than 30,000).

If desired, rather than, or in addition to, blending high and low molecular weight polylactides, a plasticizer can be added to a high molecular weight poly(lactide) or blend of high and low molecular weight poly(lactides). A plasticizer will likewise reduce the T_{g} of the polymer and can provide additional control over the T_{g}-related release kinetics of the topcoat. Plasticizers include, without limitation, cyclic lactide monomer, poly(lactic acid) oligomer, cholesterol, lecithin, diglycerides, triglycerides, fatty acids, fatty acid esters, fatty alcohols, and poly(sebacic acid-co-glycerol).

The topcoat may comprise poly(lactide) having acid end groups that, in aqueous media, will facilitate the hydrolysis of the polyester groups and thus the degradation of the polymer.

The topcoat may comprise poly(D,L-lactide) in which the ration of D to L lactide is different from that of the racemic mixture, that is, 50:50. Ratios of D:L from 5:95 to 95:5 may be used.

If desired, a diblock copolymer of poly(lactide-bl-ethylene glycol) may be used in the topcoat. A triblock copolymer, poly(lactide-bl-ethylene glycol-bl-lactide) may be used to vary the permeability and biodegradability of the topcoat.

The topcoat can also comprise a poly(D,L-lactide-co-trimethylene carbonate) copolymer, the trimethylene carbonate providing enhanced biodegradability to the layer.

The topcoat may be formulated with a quantity of the same bioactive agent in the reservoir layer or a different bioactive agent from that in the reservoir layer. This would provide an intentional burst release of the bioactive agent or a release of an activating agent that needs to have its effect prior to the administration of the reservoir layer bioactive agent.

If desired, a topcoat layer comprising a porogen can be used. A porogen is a substance that acts as a space-filling entity that is incorporated into the coating. The result of coating formation in the presence of a porogen is a bulk coating polymer containing a dispersed porogen phase which may or may not be interconnected. After coating formation is complete, the porogen must be removable. A porogen may be liquid or particulate. Examples of particulate porogens include, without limitation, sucrose, glucose, sodium chloride, phosphate salts, and ice crystals. Examples of liquid porogens include, likewise without limitation, liquids that are miscible with the coating mixture but that are inert to the coating process; liquids that form a two-phase system with the coating mixture and are likewise inert to the coating process, emulsifiers; and surfactants. As with particulate porogens, the liquid porogen must be removable from the porous network once coating is complete. By controlling the size and amount of porogens used, the ability of the resultant porous polymer to hold and then release a bioactive agent can be highly controlled.

What is claimed:

1. A method of fabricating a bioactive agent-releasing implantable medical device, comprising:
providing an implantable medical device;
providing one or more polymer(s) each of which is less than about 50 wt % crystalline at 40° C.;
providing one or more bioactive agents;
providing a first solvent or mixture of two or more solvents, each of which individually has a boiling point of about 100° C. or less at atmospheric pressure;
providing a second solvent that has, or mixture of two or more solvents each of which individually has, a boiling point at atmospheric pressure greater than 100° C. and at least one of which has a boiling point at atmospheric pressure that is at least 25° C. higher than the highest boiling first solvent at atmospheric pressure;
wherein:
each bioactive agent is at least 10% wt % soluble in the first solvent or each solvent of the first mixture of solvents; and,
each bioactive agent is less that 10% wt % soluble in the second solvent or each solvent of the second mixture of solvents;
dissolving the polymer(s) and bioactive agent(s) in a mixture of the first and the second solvent(s) at a ratio of first solvent(s) to second solvent(s) that results in a homogenous solution;
applying a layer of the homogenous solution to the medical device; and,
drying the layer of homogeneous solution to form a bioactive agent reservoir layer.

2. The method of claim 1, wherein each polymer is less than or equal to 30 wt % crystalline at 40° C.

3. The method of claim 1, wherein each polymer is less than or equal to 20 wt % crystalline at 40° C.

4. The method of claim 1, wherein each bioactive agent is less than 5 wt % soluble in the second solvent or each solvent of the second mixture of solvents.

5. The method of claim 1, wherein each bioactive agent is less than 1 wt % soluble in the second solvent or each solvent of the second mixture of solvents.

6. The method of claim 1, wherein at least one of the polymers is a poly(ester-amide).

7. The method of claim 6, wherein the poly(ester-amide) comprises:
one or more amino acid-based constitutional units;
one or more diol-based constitutional units; and,
one or more diacid-based constitutional units.

8. The method of claim 7, wherein, if an amino acid-based constitutional unit is enantiomeric, the ratio of D-amino acid to L-amino acid for each enantiometic constitutional unit is independently from about 30:70 to about 70:30.

9. The method of claim 8, wherein the ratio of D-amino acid to L-amino acid for each enantiomeric constitutional unit is about 50:50, that is, the constitutional unit is a racemate.

10. The method of claim 7, wherein the amino-acid-based consititutional unit(s) is(are) derived from L-amino acid(s).

11. The method of claim 7, wherein the amino acid-based constitutional units is (are) derived from monomers selected from the group consisting of glycine, valine, alanine, leucine, isoleucine, lysine, tyrosine, glutamic acid, cysteine and phenyalanine.

12. The method of claim 7, wherein the diol monomer-based constitutional unit(s) is (are) derived from monomers selected from the group consisting of (2C-12C)alkyldiol, (3C-8C)cycloalkyldiol; (4C-12C)alkenyldiol and (4C-12C)alkynyldiol.

13. The method of claim 7, wherein the diol-based constitutional unit(s) is (are) derived from monomers selected from the group consisting of poly(ethylene glycol), poly(propylene glycol) and hydroxy-terminated PVP.

14. The method of claim 7, wherein the diacid-based constitutional units is (are) derived from monomers selected from the group consisting of (0C-12C)alkyldiacid, (2C-12C)alkyenyldiacid, (2C-12C)alkynyldiacid and aryldiacid.

15. The method of claim 14, wherein the monomers is (are) selected from the group consisting of oxalic acid, maleic acid, malonic acid, succinic acid, adipic acid, sebacic acid, terephthalic acid and isophthalic acid.

16. The method of claim 1, wherein the polymer is selected from the group consisting of poly(L-lactide), poly(D-lactide), poly(D,L-lactide), poly(meso-lactide), poly(L-lactide-co-glycolide), poly(D-lactide-co-glycolide), poly(D,L-lactide-co-glycolide) and poly(meso-lactide-co-glycolide), wherein:
the ratio of D-lactide to L-lactide in the D,L-lactide is from about 5:95 to about 95:5.

17. The method of claim 16, wherein the ratio of D-lactide to L-lactide in the D,L-lactide is about 50:50, that is, the D,L-lactide is racemic.

18. The method of claim 1, wherein:
one or more of the first solvent(s), the second solvent(s) or both is(are) hydroscopic; and,
the homogenous solution is applied to the implantable medical device in an at least 40% relative humidity environment, wherein:
each bioactive agent is less than 10 wt % soluble in water and,
each polymer is at least 10% wt % soluble in water.

19. The method of claim 18, wherein:
the first and second solvent or mixture of solvents are identical, that is, there is effectively only one solvent or mixture of solvents and one or more of the solvent(s) is(are) hygroscopic.

20. The method of claim 18, wherein each bioactive agent is less than 5% wt % soluble in water.

21. The method of claim 18, wherein each bioactive agent is less than w/w 1 wt % soluble in water.

22. The method of claim 1, further comprising:
providing one or more topcoat polymer(s);
dissolving the topcoat polymer(s) in a solvent or mixture of solvents to form a homogenous solution;
applying the homogenous solution to the bioactive agent reservoir layer to form a solvent-containing topcoat polymer layer; and,
drying the solvent-containing polymer layer to form a topcoat layer.

23. The method of claim 22, wherein each bioactive agent is at least 10 wt % soluble in the solvent or mixture of solvents used to dissolve the topcoat polymer(s).

24. The method of claim 22, wherein each bioactive agent is less than 10 wt % soluble in the solvent or in the mixture of solvents used to dissolve the topcoat polymer(s).

25. The method of claim 22, wherein each bioactive agent is less than 5 wt % soluble in the solvent or mixture of solvents used to dissolve the topcoat polymer(s).

26. The method of claim 22, wherein each bioactive agent is less than 1 wt % soluble in the solvent or mixture of solvents used to dissolve the topcoat polymers.

27. The method of claim 22, wherein the topcoat polymer(s) is (are) selected from the group consisting of poly(L-lactide), poly(D-lactide), poly(D,L-lactide), poly(meso-lactide), poly(D,L-lactide-block-ethylene glycol-block-D,L-lactide), and poly(meso-lactide-block-ethylene glycol-block-meso-lactide) wherein:
the ratio of D-lactide to L-lactide in the D,L-lactic acid for each polymer is independently from about 30:70 to about 70:30.

28. The method of claim 27, further comprising poly(ethylene glycol) blended with the indicated polymer(s) wherein the poly(ethylene glycol) has an average molecular weight of about 1,000 Da to about 30,000 Da.

29. The method of claim 27, further comprising poly(ethylene glycol-bl-propylene glycol-bl-ethylene glycol) (Pluronic™) wherein the Pluronic™ has an average molecular weight of less than 30,000 Da.

30. The method of claim 27, wherein the ratio of D-lactide to L-lactic acid in each D,L-lactic acid-containing polymer is about 50:50.

31. The method of claim 27, wherein the topcoat polymer is poly(D,L-lactic acid).

32. The method of claim 31, wherein the poly(D,L-lactide) topcoat polymer comprises acid end groups.

33. The method of claim 27, wherein the topcoat polymer when dried forms a topcoat layer having a thickness of from about 0.1 to 20 microns.

34. The method of claim 31, wherein the poly(D,L-lactide) has an average molecular weight of from about 20,000 Da to about 500,000 Da.

35. The method of claim 34, wherein the poly(D,L-lactide has an average molecular weight of from about 20,000 Da to about 100,000 Da.

36. The method of claim 22, further comprising a plasticizer.

37. The method of claim 36, wherein the plasticizer comprises poly(D,L-lactide) having an average molecular weight of about 2,000 Da to about 20,000 Da.

38. The method of claim 22 further comprising a porogen.

39. The method of claim 1, wherein the bioactive agent comprises one or more of a therapeutic agent, a prophylactic agent and/or a diagnostic agent.

40. The method of claim 39, wherein the therapeutic or prophylactic agent is selected from the group consisting of an antiproliferative, an antineoplastic, an antiplatelet, an anticoagulant, an antifibrin, an antithrombotic, a cytostatic and an antiallergenic.

41. The method of claim 40, wherein the therapeutic or prophylactic agent is selected from the group consisting of tacrolimus, clobestasol, dexamethasone, rapamycin, 40-O-(2-hydroxyethyl)rapamycin, 40-O-(3-hydroxypropyl)rapamycin, 40-O-[2-(2-hydroxyethoxy)]ethylrapamycin and 40-O-tetrazolylrapamycin.
